(19) United States
(12) Patent Application Publication
(10) Pub. No.: US 2024/0099618 A1
(43) Pub. Date: Mar. 28, 2024

(54) Instrument Delivery System with Antithrombogenic Features

(71) Applicant: Becton, Dickinson and Company, Franklin Lakes, NJ (US)

(72) Inventors: Shelby Ann Bieritz, Durham, NC (US); Yiping Ma, Layton, UT (US)

(21) Appl. No.: 18/371,181

(22) Filed: Sep. 21, 2023

Related U.S. Application Data
(60) Provisional application No. 63/408,982, filed on Sep. 22, 2022.

(51) Int. Cl. A61B 5/15; A61L 29/04; A61L 29/08; A61L 29/10; A61L 29/14; A61L 33/00; A61M 25/06

(52) U.S. Cl. CPC A61B 5/150206; A61L 29/042; A61L 29/085; A61L 29/10; A61L 29/14; A61L 33/0041; A61M 25/0606

(57) ABSTRACT

Provided herein is a device including a housing having a proximal end, a distal end, and a sidewall therebetween defining an interior, a fluid conduit displaceably received within the housing interior and having a proximal end and a distal end, the fluid conduit having a first conduit portion at the proximal end of the fluid conduit and a second conduit portion at the distal end of the fluid conduit, wherein the fluid conduit is configured to be advanced from a first, proximal position in which the second conduit portion does not extend beyond a distal end of a catheter of the intravenous catheter assembly to a second, distal position in which the second conduit portion extends beyond a distal end of a catheter of the intravenous catheter assembly, wherein at least a portion of the fluid conduit is configured to be antithrombogenic.

### CROSS-REFERENCE TO RELATED APPLICATION

The present application claims priority to U.S. Provisional Application Ser. No. 63/408,982 entitled “Instrument Delivery System with Antithrombogenic Features” filed Sep. 22, 2022, the entire disclosure of which is hereby incorporated by reference in its entirety.

### BACKGROUND OF THE INVENTION

### Field of the Invention

Provided herein are devices and systems for use in vascular access, and, in particular, devices and systems for use with blood draw through indwelling peripheral catheters.

### Description of Related Art

A vascular access device (VAD) may access peripheral vasculature of a patient. A VAD may be indwelling for short term (days), moderate term (weeks), or long term (months to years). VADs may be used for infusion therapy and/or for blood withdrawal.

A common type of VAD is an over-the-needle peripheral intravenous catheter (PIVC). Currently, there may be several limitations to the use of a PIVC for fluid infusion or blood draw. Among these limitations are that the smaller gauge of the blood collection device that is necessary to pass through the PIVC to the patient's vasculature can result in increased dwell times due to low flow rates. This increased dwell time can result in thrombogenesis and failure of the blood collection device. Accordingly, there is a need in the art for devices and systems that allow for the introduction of instruments into a PIVC with reduced risk of thrombogenesis.

### SUMMARY OF THE INVENTION

Provided herein is a device including a housing having a proximal end, a distal end, and a sidewall therebetween defining an interior, a fluid conduit displaceably received within the housing interior and having a proximal end and a distal end, the fluid conduit having a first conduit portion at the proximal end of the fluid conduit and a second conduit portion at the distal end of the fluid conduit, wherein the fluid conduit is configured to be advanced from a first, proximal position in which the second conduit portion does not extend beyond a distal end of a catheter of the intravenous catheter assembly to a second, distal position in which the second conduit portion extends beyond a distal end of a catheter of the intravenous catheter assembly, wherein at least a portion of the fluid conduit is configured to be antithrombogenic.

In certain configurations, the fluid conduit comprises a polyurethane and/or a silicone material.

In certain configurations, at least a portion of the fluid conduit is coated with an antithrombogenic composition.

In certain configurations, the antithrombogenic composition is one or more of a hydrophilic material, heparin, a heparin mimetic, a hydrophilic lubricant, a fluorinated lubricant, and a triblock polymer.

In certain configurations, the triblock polymer comprises polyethylene oxide) (PEO) and poly(propyiene oxide) (PPO).

In certain configurations, the triblock polymer is a PEO-PPO-PEO polymer.

In certain configurations, the composition comprises poly(ethylene glycol), one or more sulfate and/or sulfonate groups, and heparin.

In certain configurations, at least a portion of the fluid conduit includes an antithrombogenic composition.

In certain configurations, the antithrombogenic composition includes a fluoro-containing moiety, a silicone moiety, and/or a poly(ethylene glycol)-containing moiety.

In certain configurations, at least a portion of the second conduit portion includes and/or is coated with an antithrombogenic composition.

In certain configurations, the second conduit portion is coated, on an inner and inner surface thereof, with an antithrombogenic composition.

In certain configurations, substantially the entire fluid conduit comprises and/or is coated with an antithrombogenic composition.

In certain configurations, the first conduit portion includes and/or is coated with an antithrombogenic composition.

In certain configurations, the connector comprises a plurality of arms configured to engage a needleless connector.

In certain configurations, the connector includes a proboscis configured to pierce a septum of the needleless connector.

Also provided herein is a catheter system, including a catheter assembly including a catheter adapter having a distal end, a proximal end, a lumen extending between the distal end and the proximal end, and a catheter secured to the catheter adapter and extending distally from the catheter adapter, wherein the catheter has a distal end and a proximal end, and a blood draw device as described herein.

In certain configurations, the catheter assembly comprises a needleless access connector, and wherein the distal end of the blood draw device housing is reversibly coupleable to the needleless access connector.

### BRIEF DESCRIPTION OF THE DRAWINGS

FIG. 1 is a perspective view of a non-limiting embodiment of a prior art catheter assembly useful with blood draw devices as described herein;

FIGS. 2A and 2B are side views of non-limiting embodiments of a blood draw device as described herein;

FIGS. 3A-3C are partial cross-sectional side views of non-limiting embodiments of a blood draw device as described herein; and

FIG. 4 is a perspective view of a non-limiting embodiment of a system including a blood draw device as described herein.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

It should be understood that any numerical range recited herein is intended to include all values and sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

Provided herein are devices and systems for introducing instruments through indwelling catheters, such as peripheral intravenous catheters (PIVCs). While certain devices (e.g., blood draw devices) are shown in the accompanying figures and described below, those of skill will appreciate that antithrombogenic features described herein may be useful in any number of different devices for introducing an instrument, including instruments ranging from tubes, probes, sensors, wiring, fiber optics, guidewires, etc.

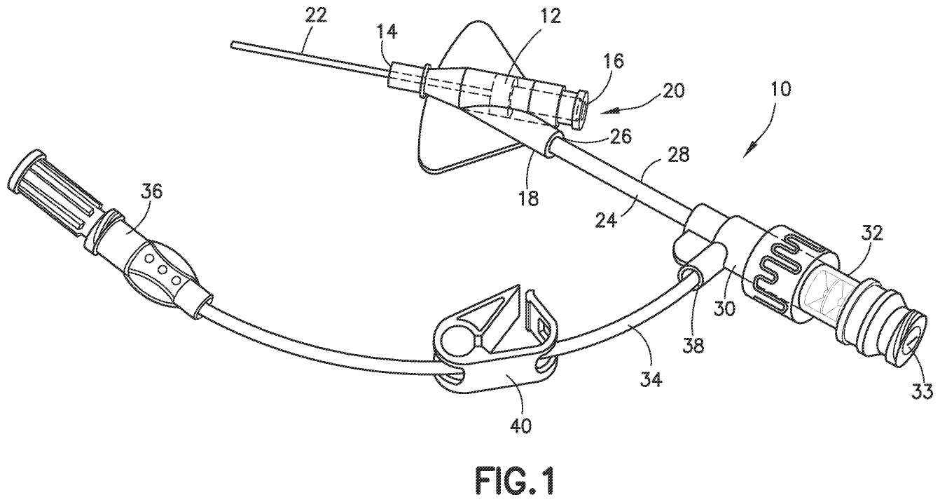

Referring now to FIG. 1, shown is a non-limiting embodiment of a catheter assembly 10 useful with blood draw devices as described herein below. Cather assembly 10 may include a catheter adapter 12, which may include a distal end 14 and a proximal end 16. In some embodiments, the catheter adapter 12 may include an additional port 18. In some embodiments, port 18 may disposed between the distal end 14 and the proximal end 16. In some embodiments, port 18 may be disposed at proximal end 16. In some embodiments, the first catheter adapter 12 may include a first lumen 20 extending through the distal end 14 and the proximal end 16. First lumen 20 may be sealed at proximal end 16 of catheter adapter 12.

In some non-limiting embodiments or aspects, the catheter assembly 10 may include a catheter 22 extending from the distal end 14. In some embodiments, the catheter 22 may include a peripheral intravenous catheter, a midline catheter, or a peripherally-inserted central catheter. Catheter 22 may be formed of any suitable material and may be of any useful length, as known to those of skill in the art. In some non-limiting embodiments or aspects, the catheter assembly 10 may include a first fluid conduit 24 extending from the port 18. First fluid conduit 24 may be formed of any suitable material known to those of skill in the art, and may have a distal end 26 and a proximal end 28, and first fluid conduit 24 may be coupled, at distal end 26 thereof, to port 18. In some non-limiting embodiments or aspects, a connector 30 may be coupled to a proximal end 28 of first fluid conduit 24. Connector 30 may be a t-connector (e.g., one side port arranged at a 90 degree angle relative to a longitudinal axis of connector 30), a y-connector (e.g., one side port arranged at a 25, a 60, or a 75 degree angle relative to a longitudinal axis of connector 30), or any other type of connector known in the art, and may include a second lumen therethrough, having any number of branches suitable for the type of connector.

In some non-limiting embodiments or aspects, catheter assembly 10 may include an extension set (integrated into or removeably coupleable to catheter adapter 12, connector 30, and/or needleless access connector 32) including a second fluid conduit, such as second fluid conduit 34. Extension sets are known to those of skill in the art and are commercially available from, for example, Becton, Dickinson and Company. In some non-limiting embodiments or aspects, second fluid conduit 34 may include a luer connection 36 at an end thereof. In some non-limiting embodiments or aspects, the extension set may include a clamp 40, to allow for occlusion of second fluid conduit 34. Clamp 40 and second fluid conduit 34 may be formed of any suitable materials known to those of skill in the art. In non-limiting embodiments, second lumen (e.g., within connector 30) has an inner diameter that is substantially equivalent to an inner diameter of first fluid conduit 24 and/or second fluid conduit 34.

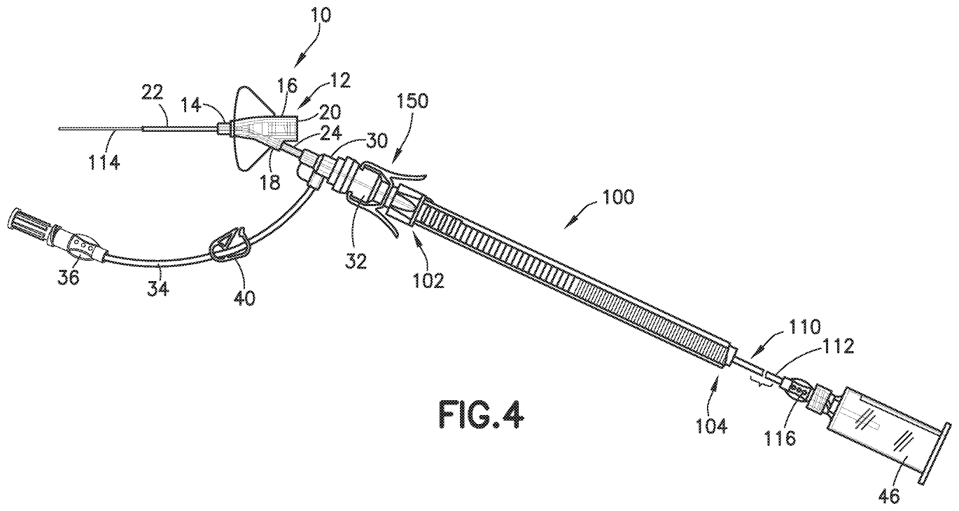

Catheter assembly 10 may include a needleless access connector 32 and/or a second fluid conduit 34. Suitable needleless access connectors 32 can include any split-septum connector, and/or those with direct fluid path access. Needleless access connectors 32 are known to those of skill in the art and are commercially available from, for example, Becton, Dickinson and Company under the tradenames MAXPLUS, MAXZERO, Q-SYTE, and SMARTSITE. Another example of a needleless access connector is the NSYTE Needle-Free Connector manufactured by NP Medical. While the non-limiting embodiments of FIGS. 1 and 4 show needleless access connectors arranged at connector 30, those of skill in the art will appreciate that suitable needleless access connectors may also be arranged at luer 36. In non-limiting embodiments, needleless access connector 32 includes a septum 33, such as a self-healing septum. In non-limiting embodiments, the septum 33 is a slit-type septum. As will be described below, a blood draw device as described herein may be reversibly coupleable to the needleless access connector 32, and one or more portions of the blood draw device may pierce the septum 33.

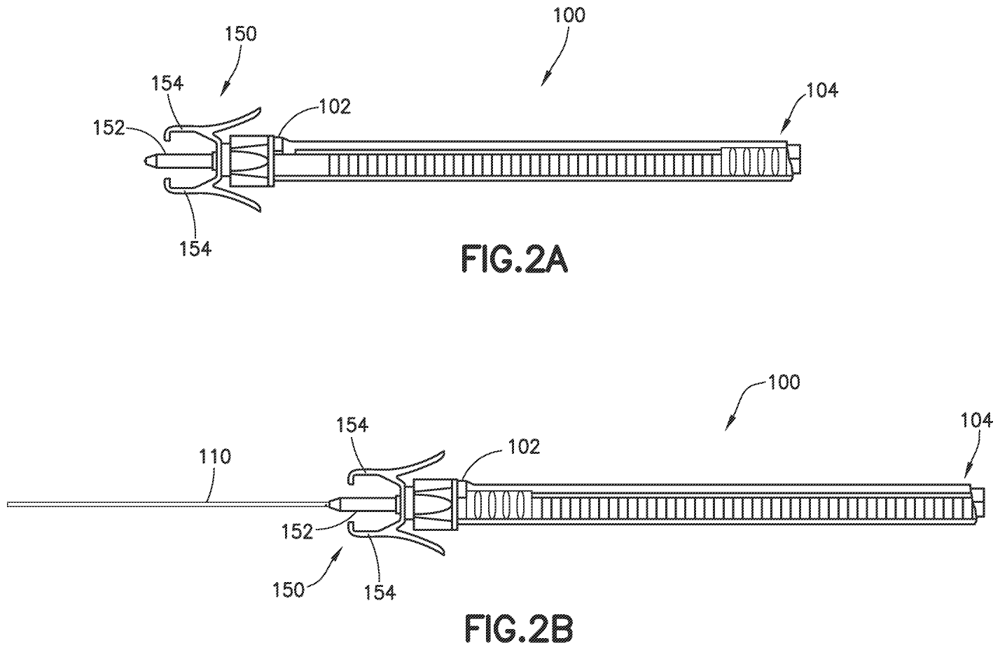

Turning to FIGS. 2A-2B, shown are non-limiting embodiments of a device for introducing an instrument. As noted above, while the illustrated embodiment is a device for introducing a fluid conduit for blood withdrawal (e.g., a blood draw device 100), those of skill will appreciate that application of antithrombogenic coatings and/or lubricants to portions of the device housing will accomplish a goal of the present invention, including reducing/preventing formation of blood clots. Exemplary blood draw devices are described in, for example, U.S. Patent Application Publication No. 2020/0316346 and U.S. Pat. No. 10,300,247, the contents of which are incorporated herein by reference in their entirety. Blood draw device 100 may include a housing 102, and, at distal end of housing 102, one or more structures for reversibly coupling to catheter assembly 10, for example via needleless access connector 32 as shown in FIG. 4. Blood draw device 100 may include, in non-limiting embodiments, at distal end of housing 102, a lock 150, including one or more arm(s) 154 and a proboscis 152, for coupling to one or more portions of catheter assembly 10. Lock 150 may be any suitable size and/or shape, and may be formed of any suitable material(s), such as plastic(s). While lock 150 is exemplified in the present figures as a plurality of arms 154 and proboscis 152, those of skill will appreciate that any connection or coupling, for example a luer, can be used, so long as distal end of housing 102 of blood draw device 100 may pass through the connection to access catheter assembly 10.

Blood draw device 100 includes a fluid flow conduit 110 received at least partially within a housing of the device 100. In non-limiting embodiments, conduit 110 is movably received within blood draw device 100, such that, when device 100 is coupled to a catheter assembly, conduit 110 is advanceable through catheter adapter 12 and catheter 22, to enter a patient's vasculature. In non-limiting embodiments, fluid flow conduit 110 is moveable within proboscis 152, such that while proboscis 152 enters a connector on catheter assembly 10, such as needleless access connector 32, fluid flow conduit 110 may move relative to proboscis 152 to access catheter assembly 10. As shown in FIG. 4, fluid flow conduit 110 may include a distal conduit portion 114 and a proximal conduit portion 112. In some embodiment, the distal conduit portion 114 and the proximal conduit portion 112 may have different inside and outside diameters.

In some embodiments, the distal conduit portion 114 may have much smaller outside diameter to allow access through catheter 22. Blood draw device 100 may include, at a proximal end of proximal conduit portion, a luer connector 116 for reversibly coupling to one or more devices to effectuate blood draw, such as a luer lock access device (LLAD), e.g., LLAD 46 as shown in FIG. 4. LLAD 46 may receive an evacuated container for withdrawing blood through conduit 110. In non-limiting embodiments, a syringe may be used to withdraw blood, by connecting such a syringe to luer connector 116 (e.g., without the need for LLAD 46).

Blood draw device 100 may include one or more antithrombogenic compositions on and/or integrated with one or more components thereof. Suitable antithrombogenic and/or anticoagulant compositions for use in medical devices that access the vasculature are known to those of skill in the art. The terms “antithrombogenic” and “anticoagulant” are used interchangeably herein, and suitable compositions can be applied to, for example in the form of a film and/or a coating, one or more surfaces of one or more components of blood draw device 100, and/or may be used in the manufacture of one or more components (e.g., antithrombogenic moieties may be included in polymeric materials used during the production of components) of blood draw device 100.

Suitable compositions include without limitation, heparin, heparin-mimetic materials, heparin, albumin, hydrophobic lubricants, fluorinated lubricants, compositions including fluoro-containing functional moieties, silicone-containing functional moieties, and/or poly(ethylene glycol) (PEG) functional moieties, antithrombogenic polymers (e.g., those containing one or both of poly(ethylene oxide) (PEO)/sulfatelsidfonate and poly(propylene oxide) (PPO), poly(2-methoxyethyl acrylate)), and combinations, phosphorylcholine, pro-drugs, and derivatives thereof. Suitable antithrombogenic compositions are also available commercially, for example the Astute® Antithrombogenic Coating sold by Biolnteractions Ltd. (Reading, UK). In non-limiting embodiments, the antithrombogenic composition includes heparin, PEG, and one or more sulfate and/or sulfonate-containing compositions. In non-limiting embodiments, the antithrombogenic composition is a PEO-PPO-PEO triblock copolymer.

In non-limiting embodiments, an antithrombogenic composition may be included with any material used to form the one or more components of blood draw device 100. For example, conduit 110 may be formed of a polymeric material, such as a polyimide material, polyurethane materials, and/or silicone material, and one or more antithrombogenic compositions may be included, as a moiety (in a polymer backbone and/or as a pendant group), therewith. Suitable compositions include those listed above, but in non-limiting embodiments include fluoro-containing functional moieties, silicone-containing functional moieties, and/or poly(ethylene glycol) (PEG) functional moieties. Suitable conduits 110 may be formed of the foregoing material by, for example, extrusion techniques. Suitable antithrombogenic compositions can be provided as films and/or coatings on any region of conduit 110, for example, on a portion of distal conduit portion 114 (including, e.g., a portion that enters the patient's vasculature), along the length of conduit 110, or only on a smaller-diameter portion of conduit 110. To this end, conduit 110 may be formed of portions having varying diameters. In order to allow for access through catheter 22, distal conduit portion 114 (optionally including one or more portions of fluid conduit 110 received within housing 102), may be of a smaller diameter than proximal conduit portion 112, which is not received within housing 102. The coatings described herein may be included on inner and/or outer surfaces of any portion of fluid conduit 110, along discrete portions, substantially the entirety of the length of conduit 110, and/or along the entirety of the length of conduit 110 (including discrete portions, substantially along the entire length of, or along the entire length of, distal portion 114, any portion of conduit 110 received within housing 102, and/or proximal portion 112).

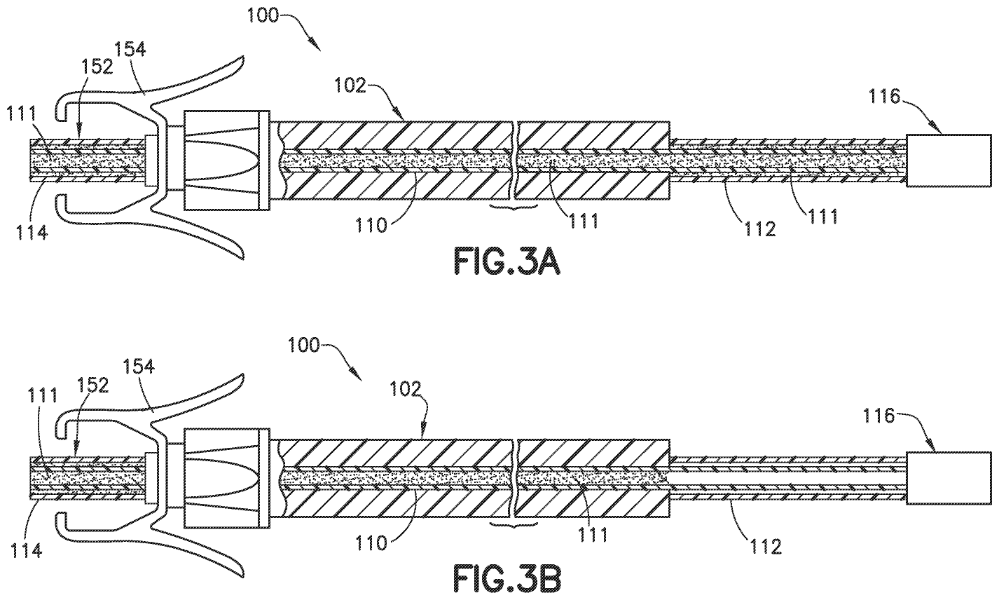

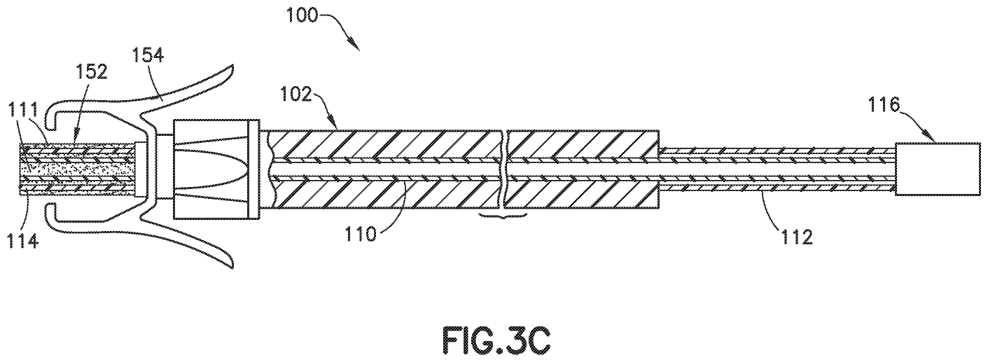

Non-limiting embodiments of the foregoing are illustrated in FIGS. 3A-3C. Specifically, FIG. 3A shows a non-limiting embodiment in which the entirety of fluid conduit 110 (including distal conduit portion 114, portion(s) of conduit 110 received within housing 102, and proximal conduit portion 112) are coated along an inner surface thereof with an antithrombogenic composition. FIG. 3B shows a non-limiting embodiment in which only an inner surface of distal conduit portion 114 and portion(s) of conduit 110 received within housing 102 is coated with an antithrombogenic composition. FIG. 3C shows a non-limiting embodiment in which only distal conduit portion 114 is coated with an antithrombogenic composition. In FIG. 3C, both inner and outer surfaces of distal conduit portion 114 are coated.

In non-limiting embodiments, antithrombogenic compositions may be included in a matrix (e.g., a cross-linked or polymerized coating) that allows for the antithrombogenic composition to be released, e.g., by virtue of being contacted with a liquid (e.g., blood) or by being exposed to one or more enzymes (e.g., in blood), as the blood draw device is received within catheter assembly 10. Suitable matrices for release, e.g., controlled release, of compositions are known to those of skill in the art and may include those disclosed in U.S. Pat. Nos. 8,821,455, 8,691,887, and 8,512,294, and U.S. Patent Application Publication Nos. 2013/0255061, 2010/0135949, and 2010/0136209, the contents of which are incorporated herein by reference in their entirety.

In non-limiting embodiments, a lubricant (including, for example, those described above) may be applied to at least a portion of a surface (e.g., an inner surface and/or an outer surface) of fluid flow conduit 110 and/or one or more portions of housing of blood draw device 100, including, without limitation, lock 150 (e.g., proboscis 152). Other suitable lubricants for medical devices that access the vasculature are known to those of skill in the art, and can include, without limitation, those disclosed in U.S. Pat. Nos. 9,956,379; 9,675,793; 8,426,348; 8,754,020; and 8,691,887, those including silicone, such as polydimethylsiloxane-containing lubricants, and like compositions.

As noted above, while the accompanying figures exemplify a blood draw device 100 with fluid flow conduit 110 that may include a coating as described above and elsewhere herein, those of skill will appreciate that other components of a device 100, such as, without limitation, wires, probes, obturators, guidewires, fibers, sensors, and like components can similarly be coated with an antithrombogenic composition.

Turning to FIG. 4, shown is a system including a catheter assembly 10 as described above, and a blood draw device 100 as also described above. In the illustrated non-limiting embodiment, blood draw device 100, through lock 150, is coupled, optionally reversibly, to needleless access connector 32 of catheter assembly 10. With reference to the non-limiting embodiment of FIG. 3C (though those of skill in the art will appreciate that the following would apply to any embodiment of the coated fluid conduit 110 or other instrument as described herein), distal conduit portion 114 may be coated with an antithrombogenic composition. When fluid conduit 110 is advanced into a portion of the catheter assembly 10, e.g. needleless access connector 32, distal conduit portion may be advanced through and beyond catheter 22. By virtue of inclusion of antithrombogenic composition as a coating on one or more surfaces of distal conduit portion 114, thrombus formation may be inhibited, allowing for blood draws to proceed without blockage caused by thrombogenesis, and without development of a thrombus in the patient's vasculature.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A blood draw device for withdrawing blood through an intravenous catheter assembly, the blood draw device comprising:
a housing comprising a proximal end, a distal end, and a sidewall therebetween defining an interior, the housing comprising a connecter at the distal end thereof for reversibly connecting the blood draw device to the intravenous catheter assembly;
a fluid conduit displaceably received within the housing interior and having a proximal end and a distal end, the fluid conduit comprising:
a first conduit portion at the proximal end of the fluid conduit; and
a second conduit portion at the distal end of the fluid conduit,
wherein the fluid conduit is configured to be advanced from a first, proximal position in which the second conduit portion does not extend beyond a distal end of a catheter of the intravenous catheter assembly to a second, distal position in which the second conduit portion extends beyond the connector and/or beyond a distal end of a catheter of the intravenous catheter assembly, and
wherein at least a portion of the fluid conduit is configured to be antithrombogenic.

2. The blood draw device of claim 1, wherein the fluid conduit comprises a polyurethane and/or a silicone material.

3. The blood draw device of claim 1, wherein at least a portion of the fluid conduit is coated with an antithrombogenic composition.

4. The blood draw device of claim 3, wherein the antithrombogenic composition is one or more of a hydrophilic material, heparin, a heparin mimetic, a hydrophilic lubricant, a fluorinated lubricant, and a triblock polymer.

5. The blood draw device of claim 3, wherein the triblock polymer comprises poly(ethylene oxide) (PEO) and poly(propylene oxide) (PPO).

6. The blood draw device of claim 3, wherein the triblock polymer is a PEO-PPO-PEO polymer.

7. The blood draw device of claim 3, wherein the composition comprises poly(ethylene glycol), one or more sulfate and/or sulfonate groups, and heparin.

8. The blood draw device of claim 1, wherein at least a portion of the fluid conduit comprises an antithrombogenic composition.

9. The blood draw device of claim 8, wherein the antithrombogenic composition comprises a fluoro-containing moiety, a silicone moiety, and/or a poly(ethylene glycol)-containing moiety.

10. The blood draw device of claim 1, wherein at least a portion of the second conduit portion comprises and/or is coated with an antithrombogenic composition.

11. The blood draw device of claim 10, wherein the second conduit portion is coated, on an inner and inner surface thereof, with an antithrombogenic composition.

12. The blood draw device of claim 1, wherein substantially the entire fluid conduit comprises and/or is coated with an antithrombogenic composition.

13. The blood draw device of claim 1, wherein the first conduit portion comprises and/or is coated with an antithrombogenic composition.

14. The blood draw device of claim 1, wherein the connector comprises a plurality of arms configured to engage a needleless connector.

15. The blood draw device of claim 14, wherein the connector comprises a proboscis configured to pierce a septum of the needleless connector.

16. A catheter system, comprising:
a catheter assembly comprising:
a catheter adapter, comprising a distal end, a proximal end, a lumen extending between the distal end and the proximal end; and
a catheter secured to the catheter adapter and extending distally from the catheter adapter, wherein the catheter comprises a distal end and a proximal end; and
a blood draw device comprising:
a housing comprising a proximal end, a distal end, and a sidewall therebetween defining an interior, the housing comprising a connecter at the distal end thereof for reversibly connecting the blood draw device to the intravenous catheter assembly;
a fluid conduit displaceably received within the housing interior and having a proximal end and a distal end, the fluid conduit comprising:
a first conduit portion at the proximal end of the fluid conduit; and
a second conduit portion at the distal end of the fluid conduit,
wherein the fluid conduit is configured to be advanced from a first, proximal position in which the second conduit portion does not extend beyond a distal end of a catheter of the intravenous catheter assembly to a second, distal position in which the second conduit portion extends beyond the connector and/or beyond a distal end of a catheter of the intravenous catheter assembly, and
wherein at least a portion of the fluid conduit is configured to be antithrombogenic.

17. The system of claim 16, wherein the catheter assembly comprises a needleless access connector, and wherein the distal end of the blood draw device housing is reversibly coupleable to the needleless access connector.
